(19) United States
(12) Patent Application Publication
(10) Pub. No.: US 2021/0275254 A1
(43) Pub. Date: Sep. 9, 2021

(54) ELECTROMAGNETIC INTERFERENCE REDUCTION IN A MEDICAL DEVICE

(71) Applicant: KONINKLIJKE PHILIPS N.V., EINDHOVEN (NL)

(72) Inventor: Willem-Jan Arend DE WIJS, OSS (NL)

(21) Appl. No.: 16/348,883

(22) PCT Filed: Nov. 16, 2017
(86) PCT No.: PCT/EP2017/079382

§ 371 Date: May 10, 2019

(30) Foreign Application Priority Data
Nov. 24, 2016 (DE) 16200430.3

(51) Int. Cl. A61B 34/20; A61B 17/34; A61B 34/00; G01S 7/52

(52) U.S. Cl. CPC A61B 34/20; A61B 17/3403; A61B 34/25; G01S 7/52077; A61B 2090/3929; A61B 2017/3413; A61B 2034/2063; A61B 2034/2072; G01S 7/52079

(57) ABSTRACT

The invention relates to a medical device having reduced susceptibility to EMI. The medical device includes a body, a first electrical conductor, a second electrical conductor, a first polarized transducer, and a second polarized transducer. The first electrical conductor and the second electrical conductor each extend along the body. The first polarized transducer and the second polarized transducer are attached to the body such that their outer faces have opposite polarity. Moreover, the first polarized transducer and the second polarized transducer are connected between the first electrical conductor and second electrical conductor either i) electrically in series and with the same polarity; or ii) electrically in parallel and with the same polarity.

### FIELD OF THE INVENTION

The present invention relates to the reduction of electromagnetic interference, EMI, in a medical device that includes a polarized transducer. The medical device may be a medical device in general and thus the invention finds application in numerous medical application areas. In one specific example the polarized transducer is an ultrasound detector that is used in tracking the position of the medical device respective the ultrasound field of a beamforming ultrasound imaging system.

### BACKGROUND OF THE INVENTION

Transducers are frequently included on medical devices in order to perform a sensing function. A sub-group of these transducers are formed from polarized, or poled, materials, i.e. materials that have an inherent polarization. When used as sensors, such polarized transducers are susceptible to electromagnetic interference from nearby electrical systems, particularly when used in a medical environment.

One example of a polarized transducer is a piezoelectric ultrasound detector. Piezoelectric materials such as lead zirconium titanate, i.e. PZT, polyvinylidene fluoride, i.e. PVDF, and lithium niobate are commonly used in ultrasound detection and have an inherent polarization. When disposed in an ultrasound field the ultrasound vibrations result in a change in their surface charge. An electrical circuit connected to the material is used to sense the surface charge and thereby detect ultrasound. Electromagnetic interference from nearby electrical systems can limit the performance of such a sensor by degrading its ability to detect weak ultrasound signals. Polarized transducers may also be formed from other materials such as pyroelectric and ferroelectric materials. Such materials may be used to form sensors of e.g. infrared radiation, temperature, pressure, and sound, i.e. a microphone. These polarized transducers may likewise suffer from EMI.

One exemplary medical device in which it is desirable to reduce EMI is an ultrasound-based tracking system disclosed in patent application WO/2011/138698. In this system the position of a medical device is tracked respective the ultrasound field of a beamforming ultrasound imaging system based on ultrasound signals transmitted between the ultrasound probe and an ultrasound detector attached to the medical device. The position of the medical device is determined by correlating ultrasound signals emitted by the ultrasound probe with those detected by the ultrasound detector on the medical device. The ultrasound detector may for example be a polarized transducer formed from a piezoelectric material. In such a system the reduction of EMI is important in maintaining the accuracy of the tracking system.

Document WO2015/155649 also relates to an ultrasound-based tracking system for tracking a medical device. In this system a polarized ultrasound detector is likewise used to detect ultrasound signals. EMI is reduced by locating a dummy detector adjacent the tracking detector and determining the position of the medical device based on the difference between the electrical signals generated by the two detectors.

Document US 2009/230820A1 discloses a piezoelectric transducer formed of a body of piezoelectric material having first and second opposed sides and first and second electrically conductive layers on the first and second sides respectively of the piezoelectric body, wherein the piezoelectric body and the electrically conductive layers are so constructed that they form a plurality of separate adjacent series-connected transducer elements. The piezoelectric body may have a substantially uniform direction of polarization, or alternating zones of opposite polarization. The elements can be hard wired or connected through a switching circuit to display either circumferential or axial or other ultrasonic focal patterns, and may be connected in a parallel, rather than a series configuration.

Document U.S. Pat. No. 5,298,828A discloses an ultrasonic transducer that has a pair of transducer elements polarised in opposite directions, which are mounted between, and in intimate contact with, respective front face electrodes and back face electrodes. The front face electrodes are each earthed. The back face electrodes are each connected to a respective input/output terminal. The input/output terminals are supplied with activating pulses of opposite polarity, produced using a differential pulse generator or a transformer arrangement, when the transducer is operating in the transmit mode. When the transducer is operating in the receive mode, pulses of opposite polarity are generated at the back face electrodes when an ultrasonic pressure wave is incident upon the front face electrodes. These pulses are differentially summed using a differential amplifier or a transformer arrangement. Such a transducer has a substantially reduced pick-up of environmental noise and thus has an improved signal to noise ratio when in use.

Document WO 2015/155645 A1 discloses a medical device that includes a conductive body including a surface and a sensor conformally formed on the surface and including a piezoelectric polymer formed about a portion of the surface and following a contour of the surface. The piezoelectric polymer is configured to generate or receive ultrasonic energy.

However, there remains a need to further reduce EMI in medical devices that include a polarized transducer.

### SUMMARY OF THE INVENTION

It is an object of the present invention to reduce EMI in a medical device that includes a polarized transducer. Thereto, a medical device, a position tracking system, a software-implemented method of discriminating between ultrasound signals and electromagnetic interference, and a transducer laminate for attachment to the shaft of a medical device are provided.

In accordance with one aspect the medical device includes a body, a first electrical conductor, a second electrical conductor, a first polarized transducer, and a second polarized transducer. The first electrical conductor and the second electrical conductor each extend along the body. The first polarized transducer and the second polarized transducer are attached to the body such that their outer faces have opposite polarity. Moreover, the first polarized transducer and the second polarized transducer are connected between the first electrical conductor and second electrical conductor either i) electrically in series and with the same polarity; or ii) electrically in parallel and with the same polarity.

In so doing a medical device with a polarized transducer is provided in which a common EMI signal is picked-up by the first electrical conductor and the second electrical conductor. The common EMI signal can be removed by subsequently subtracting the electrical signals on the first electrical conductor and the second electrical conductor. This may be achieved by differential amplification of the signals. At the same time the above electrical connection provides a useful transducer signal, thereby retaining the transducer's desired sensing functionality.

In accordance with another aspect a position tracking system is provided. The position tracking system includes an ultrasound imaging probe, an image reconstruction unit, a position determination unit, the above-described medical device in which the first polarized transducer and the second polarized transducer are each configured to detect ultrasound signals, a differential amplifier circuit, and an icon providing unit. The ultrasound imaging probe is configured to generate and to detect ultrasound signals within an ultrasound field. The image reconstruction unit is configured to provide, based on the ultrasound signals generated by and detected by the ultrasound imaging probe, a reconstructed ultrasound image corresponding to the ultrasound field. The differential amplifier circuit is electrically connected to the first electrical conductor and to the second electrical conductor of the medical device and is configured provide, in response to the detection of ultrasound signals transmitted between the ultrasound imaging probe and the medical device, an amplified difference electrical signal corresponding to an amplified difference between an electrical signal carried by the first electrical conductor and an electrical signal carried by the second electrical conductor. The position determination unit is configured to receive the amplified difference electrical signal, and to compute, based on the amplified difference electrical signal and based on the ultrasound signals transmitted between the ultrasound imaging probe and the medical device, a position of the medical device respective the ultrasound field. Moreover the icon providing unit is configured to provide, in the reconstructed image, an icon indicating the position of the medical device respective the ultrasound field. In so doing a position tracking system with reduced EMI is provided. Consequently, the accuracy of the position tracking is improved.

In accordance with another aspect a software-implemented method of discriminating between ultrasound signals and electromagnetic interference is provided. The method includes the steps of i) causing amplification, with a differential amplifier circuit, of a difference between an electrical signal carried by the first electrical conductor and an electrical signal carried by the second electrical conductor of the medical device of claim 1 wherein the first polarized transducer and the second polarized transducer are configured to detect ultrasound signals to provide an amplified difference electrical signal, ii) causing conversion, with an analogue to digital converter circuit, of the amplified difference electrical signal into a digital signal. In so doing a digital signal corresponding to a detected ultrasound signal is provided with reduced EMI.

In accordance with another aspect a transducer laminate is provided for attachment to the shaft of a medical device. The medical device may for example be a needle. The transducer laminate comprises a first elongate foil, a second elongate foil, a first electrical conductor, a second electrical conductor, a first polarized transducer for detecting ultrasound signals, and a second polarized transducer for detecting ultrasound signals. The first elongate foil, the second elongate foil, the first electrical conductor and the second electrical conductor each extend along a length axis. At a first position along the length axis the first electrical conductor, the second electrical conductor, the first polarized transducer and the second polarized transducer are sandwiched between the first elongate foil and the second elongate foil. The first polarized transducer and the second polarized transducer are arranged adjacent to one another and such that their outer faces that face the first elongate foil have opposite polarity. Moreover, the first polarized transducer and the second polarized transducer are connected between the first electrical conductor and second electrical conductor either i) electrically in series and with the same polarity; or ii) electrically in parallel and with the same polarity. Furthermore, at a second position along the length axis the first electrical conductor and the second electrical conductor are sandwiched between the first elongate foil and the second elongate foil and neither the first polarized transducer nor the second polarized transducer are sandwiched between the first elongate foil and the second elongate foil. In so doing a transducer laminate is provided that is less susceptible to EMI. The transducer laminate may be easily attached to a medical device and therefore simplifies its manufacture.

Other aspects are defined in the dependent claims.

### BRIEF DESCRIPTION OF THE DRAWINGS

FIG. 1 illustrates a medical device MD including first polarized transducer PT1, second polarized transducer PT2, first electrical conductor EC1, and second electrical conductor EC2.

FIG. 2 illustrates various electrical circuits that include first polarized transducer PT1, second polarized transducer PT2, first electrical conductor EC1, and second electrical conductor EC2 and which do not fall within the scope of the invention.

FIG. 3 illustrates a medical device MD that includes first polarized transducer PT1, second polarized transducer PT2, first electrical conductor EC1, and second electrical conductor EC2 in which the first electrical conductor EC1, and second electrical conductor EC2 are electrically connected to optional differential amplifier circuit DACCT.

FIG. 4 illustrates a position tracking system PTS that includes an ultrasound imaging system UIS and a medical device MD.

FIG. 5 illustrates a transducer laminate TL that may be attached to a shaft of a medical device.

### DETAILED DESCRIPTION OF THE INVENTION

In order to illustrate the principles of the present invention, various medical devices that have reduced susceptibility to EMI are described. Although the medical devices are exemplified by a needle, it is to be appreciated that the invention also finds application in other medical devices such as a catheter, a guidewire, a probe, an endoscope, an electrode, a robot, a filter device, a balloon device, a stent, a mitral clip, a left atrial appendage closure device, an aortic valve, a pacemaker, an intravenous line, a drainage line, a surgical tool, a tissue sealing device, or a tissue cutting device.

Moreover, the medical device is described in relation to a position tracking system in which the position of the medical device is determined based on ultrasound signals detected by polarized transducers attached to the medical device. Although the position tracking system includes a 2D ultrasound imaging probe in which the position of the medical device is determined in relation to an image plane that is generated by the 2D ultrasound imaging probe, the medical device also finds application in position tracking systems that use other types of imaging probes, including a 3D imaging probe, a “TRUS” transrectal ultrasonography probe, an “IVUS” intravascular ultrasound probe, a “TEE” transesophageal probe, a “TTE” transthoracic probe, a “TNE” transnasal probe, an “ICE” intracardiac probe. More generally, it is to be appreciated that these position tracking systems are purely used as example applications in which the medical device may be used, and that the medical device may also find application in a wide range of sensing applications that include polarized transducers. These include, but are not limited to sensors of temperature, radiation, pressure, sound, ultrasound and so forth.

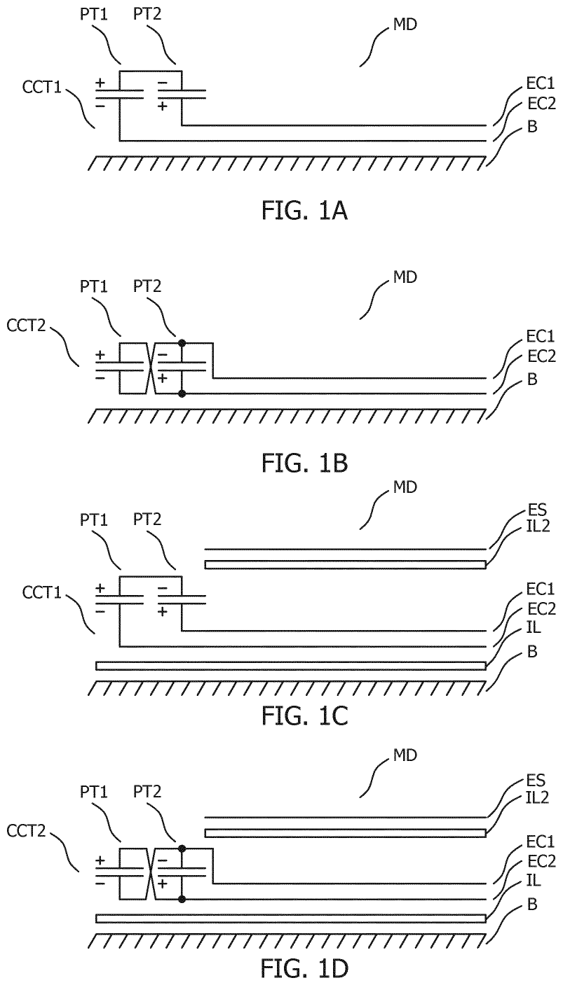

FIG. 1 illustrates a medical device MD including first polarized transducer PT1, second polarized transducer PT2, first electrical conductor EC1, and second electrical conductor EC2. Medical device MD in FIG. 1 has a body, B, and may for example be a medical needle in which the needle shaft is represented by body B. Optionally, body B may be formed from a conductor. The polarized transducers PT1, PT2 in FIG. 1 may for example be ultrasound detectors formed from PVDF material. In FIG. 1 first electrical conductor EC1 and second electrical conductor EC2 each extend along body B. First electrical conductor EC1 and second electrical conductor EC2 may for example be formed from a metal such as aluminium or gold. Alternatively a range of conductive materials such as inks, adhesives and polymers may be used. Moreover, in FIG. 1 first polarized transducer PT1 and second polarized transducer PT2 are attached to the body B such that their outer faces have opposite polarity. Thus as illustrated in FIG. 1 first polarized transducer PT1 has its positive electrode facing outwards with respect to body B, and second polarized transducer PT2 has its negative electrode facing outwards with respect to the surface of body B. The positive and negative electrodes of the transducer correspond to a polling direction in the transducer. In other words, each polarized transducer PT1, PT2 has a polling direction and the polling directions of each of polarized transducers PT1, PT2 are oppositely arranged with respect to the surface of body B. Moreover, in FIG. 1 first polarized transducer PT1 and second polarized transducer PT2 are electrically connected between the first electrical conductor EC1 and the second electrical conductor EC2.

FIG. 1A illustrates a first embodiment of a medical device MD in which first polarized transducer PT1 and second polarized transducer PT2 are connected between the first electrical conductor EC1 and the second electrical conductor EC2 electrically in series and with the same polarity CCT1. In other words the polarities of the first polarized transducer PT1 and the second polarized transducer PT2 are additive. In the arrangement of FIG. 1A, EMI from various external sources may be picked-up by each of the outer electrodes of first polarized transducer PT1 and second polarized transducer PT2. It has been found that this EMI pickup by the outer electrodes can indeed be higher than the EMI pickup by electrical conductors EC1, EC2. EMI that is picked-up by the outer electrode of first polarized transducer PT1 couples via the inherent capacitance of first polarized transducer PT1 to second electrical conductor EC2. This results in an interference signal on second electrical conductor EC2. Likewise, EMI that is picked-up by the outer electrode of second polarized transducer PT2 couples via the inherent capacitance of second polarized transducer PT2 to first electrical conductor EC1. This results in an interference signal on first electrical conductor EC1. Because interference signals are present on each of first electrical conductor EC1 and second electrical conductor EC2, any interference that is common to both of these electrical conductors can be removed by subsequently subtracting, i.e. differencing, the electrical signals on these conductors, for example by differentially amplifying them. The series electrical connection of CCT1 also ensures that ultrasound signals detected by each of first polarized transducer PT1 and second polarized transducer PT2 generate electrical signals that do not cancel one another. Thus, a useful transducer signal can be detected by the embodiment of FIG. 1A with the benefit of reduced EMI. Although not essential to the reduction of EMI, preferably the inherent capacitance of first polarized transducer PT1 and second polarized transducer PT2 are similar, or equal, and/or preferably the outer faces of each of first polarized transducer PT1 and second polarized transducer PT2 have a similar or equal area. Thus the arrangement in FIG. 1A, provides reduced EMI as compared to a reference circuit in which a single polarized transducer is in electrical connection with electrical connectors EC1, EC2. This is because there is a common interference signal present on each of first electrical conductor EC1 and second electrical conductor EC2.

FIG. 1B illustrates a second embodiment of a medical device MD in which first polarized transducer PT1 and second polarized transducer PT2 are connected between the first electrical conductor EC1 and the second electrical conductor EC2 electrically in parallel and with the same polarity CCT2. In other words the positive polarity electrodes of each polarized transducer share a common positive electrical node, and the negative polarity electrodes of each polarized transducer PT1, PT2 share a common negative electrical node. As in the embodiment of FIG. 1A, in the arrangement of FIG. 1B, EMI from various external sources may by picked-up by each of the outer electrodes of first polarized transducer PT1 and second polarized transducer PT2. In FIG. 1B, EMI that is picked-up by the outer electrode of first polarized transducer PT1 couples via the inherent capacitance of first polarized transducer PT1 to first electrical conductor EC1. This results in an interference signal on first electrical conductor EC1. Likewise, EMI that is picked-up by the outer electrode of second polarized transducer PT2 couples via the inherent capacitance of second polarized transducer PT2 to second electrical conductor EC2. This results in an interference signal on second electrical conductor EC2. Because interference signals are present on each of first electrical conductor EC1 and second electrical conductor EC2, any interference that is common to both of these electrical conductors can be removed by subsequently subtracting, i.e. differencing, the electrical signals on these conductors, for example by differentially amplifying them. The parallel electrical connection of CCT2 also ensures that ultrasound signals detected by each of first polarized transducer PT1 and second polarized transducer PT2 generate electrical signals that do not cancel one another. Thus, a useful signal can be detected by the embodiment of FIG. 1B with the benefit of reduced EMI. Although not essential to the reduction of EMI, preferably the inherent capacitance of first polarized transducer PT1 and second polarized transducer PT2 are similar, or equal, and/or preferably the outer faces of each of first polarized transducer PT1 and second polarized transducer PT2 have a similar or equal area. Thus the arrangement in FIG. 1B provides reduced EMI as compared to a reference circuit in which a single polarized transducer is in electrical connection with electrical connectors EC1, EC2. This is because there is a common interference signals present on each of first electrical conductor EC1 and second electrical conductor EC2.

FIG. 1C and FIG. 1D illustrate third and fourth embodiments of a medical device MD that correspond to those of FIG. 1A and FIG. 1B respectively, and which additionally include optional electrical shield ES and optional insulator layers IL, IL2. Electrical shield ES sandwiches at least a portion of the first electrical conductor EC1 and a portion of the second electrical conductor EC2 between the electrical shield ES and the body B. Depending on the relative sizes of electrical conductor EC1 and second electrical conductor EC2 in relation to the surface area of first polarized transducer PT1 and second polarized transducer PT2, significant EMI can also be picked-up by these electrical conductors. Electrical shield ES may thus serve to reduce the EMI picked-up by these electrical conductors. Optionally electrical shield ES may be electrically connected to body B; for example in the vicinity of PT1, PT2, or along the length of electrical shield ES in order to further reduce EMI coupling. Optional insulator layer IL2 is disposed between electrical shield ES and a portion of the first electrical conductor EC1 and a portion of the second electrical conductor EC2. Insulator layer IL2 may serve to improve electrical isolation between electrical shield ES and the electrical conductors EC1, EC2. Optionally electrical shield ES and/or insulator layer IL2, may also cover a portion or all of first polarized transducer PT1 and/or second polarized transducer PT2 in order to further reduce the coupling of EMI to electrical conductors EC1, EC2. Electrical shield ES may be formed from a range of conductive materials such as a metal, for example aluminium or gold, indium tin oxide, ITO, conductive polymers and so forth. Insulator layer IL in FIG. 1C and FIG. 1D is disposed between body B and the first polarized transducer PT1, the second polarized transducer PT2, the first electrical conductor EC1 and the second electrical conductor EC2. Alternatively Insulator layer IL in FIG. 1C and FIG. 1D may be disposed only between body B and first electrical conductor EC1 and second electrical conductor EC2, or only between body B and the first polarized transducer PT1 and the second polarized transducer PT2. Insulator layer IL serves to improve electrical isolation between body B and each of the electrical conductors EC1, EC2, and between body B and each of polarized transducers PT1, PT2. Insulator layer IL may be particularly useful in reducing EMI if body B is formed from an electrically conductive material, such as a metal, for example the stainless steel shaft of a medical needle. Insulator layer IL may for example be formed from a polymer, a ceramic, a dielectric material and so forth.

Clearly the polarity of the outer electrodes of both first polarized transducer PT1 and second polarized transducer PT2 in FIG. 1 may be reversed to achieve the same benefits.

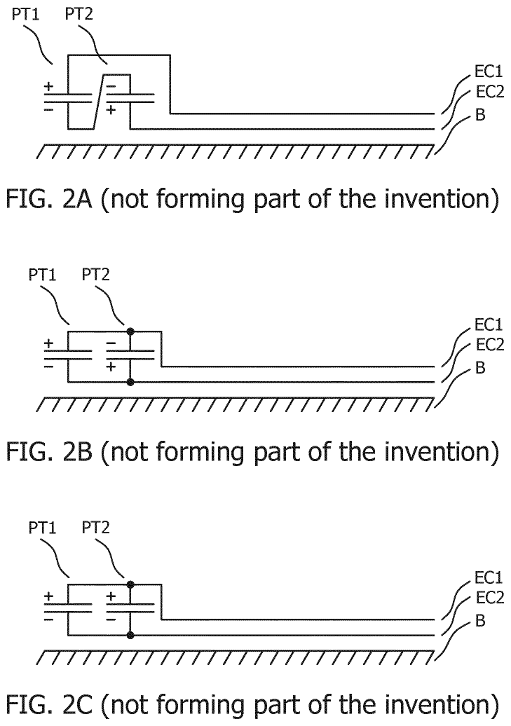

FIG. 2 illustrates various electrical circuits that include first polarized transducer PT1, second polarized transducer PT2, first electrical conductor EC1, and second electrical conductor EC2 and which do not fall within the scope of the invention. FIG. 2A and FIG. 2B are inappropriate since the electrical signals generated by each of first polarized transducer PT1 and the second polarized transducer PT2 counteract one another. FIG. 2C benefits from additional transducer signal due to the use of two polarized transducers PT1, PT2 but is inappropriate since it does not benefit from the EMI reduction mechanism described above.

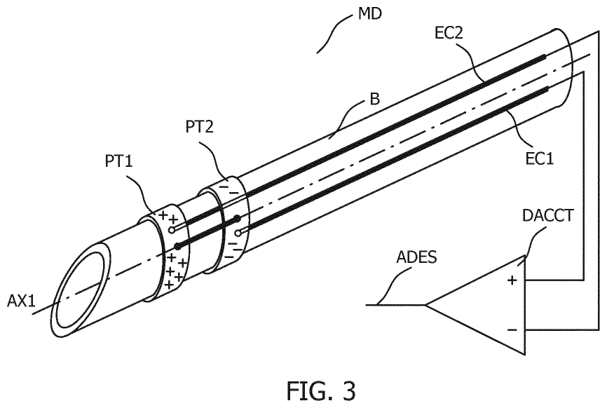

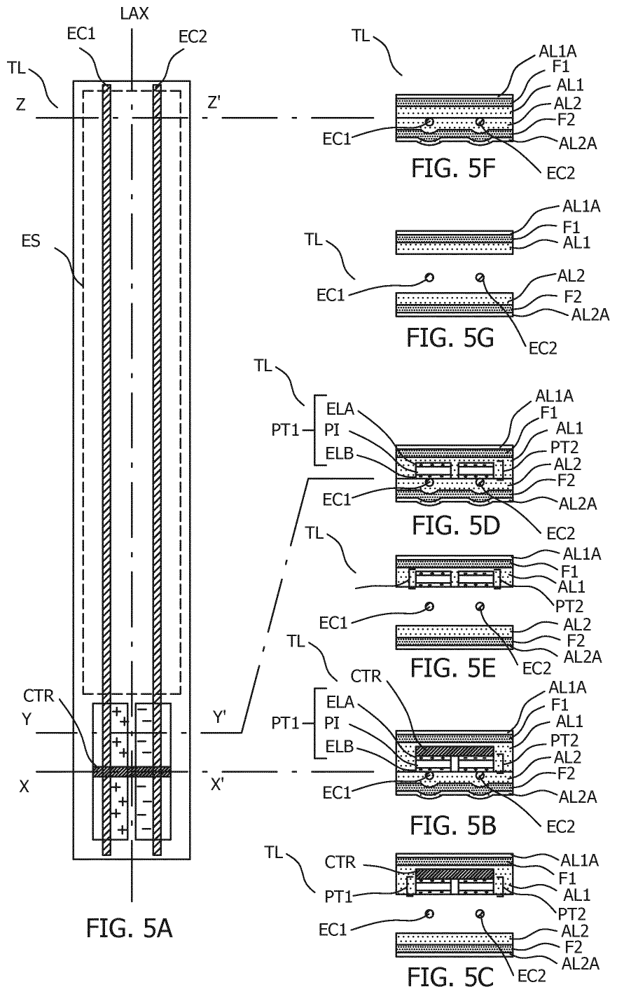

FIG. 3 illustrates a medical device MD that includes first polarized transducer PT1, second polarized transducer PT2, first electrical conductor EC1, and second electrical conductor EC2 in which the first electrical conductor EC1, and second electrical conductor EC2 are electrically connected to optional differential amplifier circuit DACCT. Medical device MD in FIG. 3 is illustrated as a medical needle having a body B that is the shaft of the medical needle. As described above, other medical devices may alternatively be used. The electrical circuit used in FIG. 3 corresponds to CCT1 in FIG. 1A although CCT2 in FIG. 1B may alternatively be used. Either of these electrical circuits CCT1, CCT2 may be used optionally in combination with electrical shield ES and optional insulator layer IL as illustrated in FIG. 1C and FIG. 1D. First polarized transducer PT1 and second polarized transducer PT2 are preferably ultrasound transducers although any other polarized transducer may alternatively be used, such as the example transducers described herein. The electrical interconnections between first polarized transducer PT1, second polarized transducer PT2 may be made using conventional electrical interconnection techniques such as wire bonding, conductive adhesives, soldering and so forth. Alternatively a transducer laminate described later with reference to FIG. 5 may be used in which the desired electrical connections may be provided via pressure contact.

Medical device MD in FIG. 3 has a body B that has an elongate form and an axis AX1. Other shapes of body B may alternatively be used. First electrical conductor EC1 and second electrical conductor EC2 in FIG. 3 each extend along axis AX1. First electrical conductor EC1 and second electrical conductor EC2 are preferably formed from a wire since a wire is robust to bending and wrapping processes. Alternatively other shapes of electrical conductors such as electrical tracks may be used. EMI to the electrical conductors may be reduced by providing a similar path for each of the electrical conductors EC1, EC2. Thereto the paths of electrical conductors EC1, EC2 are preferably arranged parallel to one another. In one configuration first electrical conductor EC1 and second electrical conductor EC2 may each be wrapped around the body in the form of a spiral. As described later, such spiral wrapping has the additional benefit of simplifying a laminate that can be attached to such an elongate device. As illustrated in FIG. 3 first polarized transducer PT1 and second polarized transducer PT2 are wrapped around the elongate body in the form of a ring. Such a ring configuration provides sensing around axis AX1 of medical device MD without medical device MD obscuring the transducer. Moreover in FIG. 3 first polarized transducer PT1 and second polarized transducer PT2 are separated along the axis AX1. Alternatively first polarized transducer PT1 and second polarized transducer PT2 may be disposed adjacent to one another; i.e. in a side-by-side configuration around a circumference of body B about axis AX1.

Optional differential amplifier circuit DACCT in FIG. 3 is electrically connected to first electrical conductor EC1 and second electrical conductor EC2 and is configured to generate an amplified difference electrical signal ADES corresponding to an amplified difference between an electrical signal carried by the first electrical conductor EC1 and an electrical signal carried by the second electrical conductor EC2. The modulus of the gain of differential amplifier circuit DACCT may for example be greater than or equal to unity. Many suitable differential amplifier circuits known from the electronics field may be used for this depending on the type of electrical signal; i.e. a charge, a voltage, or a current that is generated by the corresponding polarized transducer PT1, PT2. Differential amplifier circuit DACCT provides an amplified difference electrical signal ADES corresponding to an amplified difference between an electrical signal carried by first electrical conductor EC1 and an electrical signal carried by second electrical conductor EC2. Amplified difference electrical signal ADES may subsequently be further processed by electronic circuits, for example converted into a digitized form using a digital to analog converter, or DAC, circuit.

Optionally a processor may be provided in order to control the process of amplification by the differential amplifier circuit DACCT and the process of conversion of its amplified difference electrical signal ADES into a digital signal. The processor may thus execute a software-implemented method of discriminating between transducer signals and electromagnetic interference. The software-implemented method may be stored on a computer program product as instructions that are executable by the processor. The computer program product may be provided by dedicated hardware, or hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor “DSP” hardware, read only memory “ROM” for storing software, random access memory “RAM”, non-volatile storage, etc. Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system, or apparatus or device, or a propagation medium. Examples of a computer-readable medium include a semiconductor or solid state memory, magnetic tape, a removable computer diskette, a random access memory “RAM”, a read-only memory “ROM”, a rigid magnetic disk and an optical disk. Current examples of optical disks include compact disk read only memory “CD-ROM”, compact disk read/write “CD-R/W”, Blu-Ray™ and DVD.

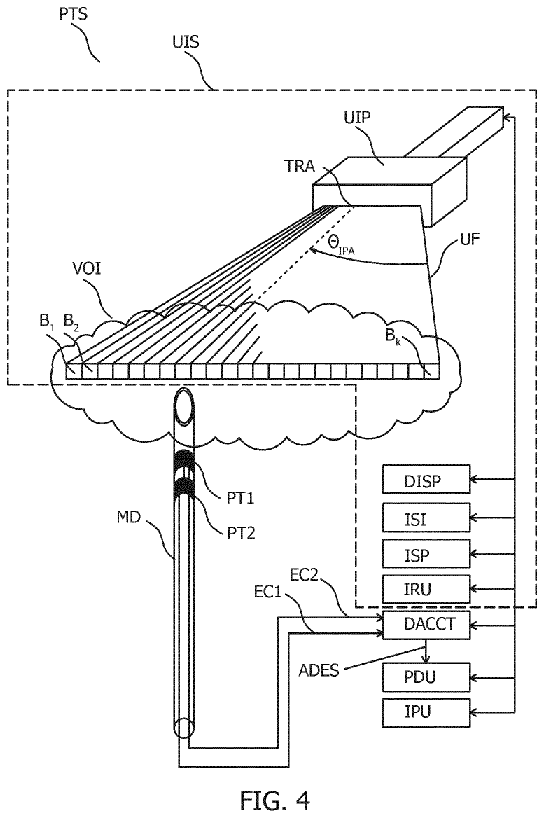

The above-described medical device finds application in a wide range of applications. One such exemplary application is a position tracking system for tracking a position of a medical device MD based on ultrasound signals. In this application, first polarized transducer PT1 and second polarized transducer PT2 of the medical device MD are each configured to detect ultrasound signals. FIG. 4 illustrates a position tracking system PTS that includes an ultrasound imaging system UIS and a medical device MD.

In FIG. 4, ultrasound imaging system UIS includes ultrasound imaging probe UIP, image reconstruction unit IRU, imaging system processor ISP, imaging system interface ISI and display DISP. The units in FIG. 4 are in communication with each other as indicated by the interconnecting arrows. Ultrasound imaging system UIS corresponds to a conventional ultrasound imaging system. Units IRU, ISP, ISI and DISP are conventionally located in a console that is in wired communication with ultrasound imaging probe UIP. It is also contemplated that wireless communication, for example using an optical, infrared, or an RF communication link, may replace the wired link. It is also contemplated that some of units IRU, ISP, ISI and DISP may instead be incorporated within ultrasound imaging probe UIP, as in for example the Philips Lumify ultrasound imaging system.

In FIG. 4, ultrasound imaging probe UIP includes linear ultrasound transceiver array TRA that transmits and receives ultrasound energy within ultrasound field UF. Ultrasound field UF intercepts volume of interest VOI. Ultrasound field UF is fan-shaped in FIG. 4 and is defined by ultrasound beams B_{1 . . . k}. Note that although a fan-shaped beam is illustrated in FIG. 4 the tracking of the medical device MD is not limited to a particular shape of ultrasound field; for example a 3D field may also be used. Ultrasound imaging probe UIP may also include electronic driver and receiver circuitry not shown that is configured to amplify and/or to adjust the phase of signals transmitted by or received by ultrasound imaging probe UIP in order to generate and detect ultrasound signals in beams B_{1 . . . k}. The electronic driver and receiver circuitry may thus be used to steer the emitted and/ or received ultrasound beam direction.

In-use, ultrasound imaging system UIS in FIG. 4 is operated in the following way. An operator may plan an ultrasound procedure via imaging system interface ISI. Once an operating procedure is selected, imaging system interface ISI triggers imaging system processor ISP to execute application-specific programs that generate and interpret the signals generated by and detected by ultrasound imaging probe UIP. Ultrasound imaging system UIS may also include a memory, not shown, for storing such programs. The memory may for example store ultrasound beam control software that is configured to control the sequence of ultrasound signals generated by and/or detected by ultrasound imaging probe UIP. Image reconstruction unit IRU, whose function may alternatively be carried-out by imaging system processor ISP, reconstructs data received from ultrasound imaging probe UIP into an image corresponding to ultrasound field UF, and subsequently displays this image on display DISP. The reconstructed image may for example be an ultrasound Brightness-mode “B-mode” image, otherwise known as a “2D mode” image, a “C-mode” image or a Doppler mode image, or indeed any ultrasound image.

Also shown in FIG. 4 is medical device MD in the form of an exemplary medical needle, together with differential amplifier circuit DACCT, position determination unit PDU and icon providing unit IPU. Whilst illustrated as separate units it is also contemplated that the function of one or more of units PDU and IPU may be carried out within ultrasound imaging system UIS, for example within a memory or a processor that provides the functionality of units IRU and ISP.

First polarized transducer PT1 and second polarized transducer PT2 attached to medical device MD are each configured to detect ultrasound signals. Differential amplifier circuit DACCT is electrically connected to first electrical conductor EC1 and to second electrical conductor EC2 of medical device MD and is configured provide, in response to the detection of ultrasound signals transmitted between ultrasound imaging probe UIP and medical device MD, an amplified difference electrical signal ADES corresponding to an amplified difference between an electrical signal carried by first electrical conductor EC1 and an electrical signal carried by second electrical conductor EC2. Any EMI that is common to both first electrical conductor EC1 and second electrical conductor EC2 is thus cancelled in signal ADES. Position determination unit PDU is configured to receive amplified difference electrical signal ADES, and to compute, based on this signal, and based on ultrasound signals transmitted between the ultrasound imaging probe and the medical device, a position of the medical device respective the ultrasound field.

In the configuration illustrated in FIG. 4 the position of the medical device is determined based on ultrasound signals generated by ultrasound transceiver array TRA of ultrasound imaging probe UIP which are subsequently detected by polarized transducers PT1, PT2; i.e. transmitted between ultrasound imaging probe UIP and medical device MD. In this configuration, polarized transducers PT1, PT2 each receive ultrasound signals corresponding to beams B_{1 . . . k}. Polarized transducers PT1, PT2 may be electrically connected as in either of the electrical circuits CCT1, CCT2 described with reference to FIG. 1. Amplified difference electrical signal ADES includes a signal corresponding to the ultrasound signals generated by ultrasound transceiver array TRA. Position determination unit PDU identifies the mean position of polarized transducers PT1, PT2 by correlating the ultrasound signals generated by ultrasound transceiver array TRA with ultrasound signals detected by polarized transducers PT1, PT2. More specifically this correlation determines the best fit position of polarized transducers PT1, PT2 respective ultrasound field UF based on i) the amplitudes of the ultrasound signals corresponding to each beam B_{1 . . . k} that are detected by polarized transducers PT1, PT2 and ii) based on the time of flight between generation of each beam B_{1 . . . k} and its detection by polarized transducers PT1, PT2. This may be illustrated as follows. When polarized transducers PT1, PT2 are the vicinity of ultrasound field UF, ultrasound signals from the nearest of beams B_{1 . . . k} to polarized transducers PT1, PT2 will be detected with a relatively larger amplitude whereas more distant beams will be detected with relatively smaller amplitudes. Typically the beam that is detected with the largest amplitude is identified as the one that is closest to the mean position of polarized transducers PT1, PT2. This defines the in-plane angle Θ_{IPA} between ultrasound transceiver array TRA and the mean position of polarized transducers PT1, PT2. The range between the respective emitter in ultrasound transceiver array TRA and the mean position of polarized transducers PT1, PT2 is determined from the time of flight between the generation of the largest-amplitude beam B_{1 . . . k} and its subsequent detection. The range is determined by multiplying the time of flight by the speed of ultrasound propagation. Thus, the range and the in-plane angle identify the best-fit position of the mean position of polarized transducers PT1, PT2 respective ultrasound field UF.

In another configuration not illustrated in FIG. 4, ultrasound imaging probe UIP further includes at least three ultrasound emitters that are attached to the ultrasound imaging probe UIP. The at least three ultrasound emitters are in communication with position determination unit PDU. In this configuration the ultrasound field UF is again used to provide an ultrasound image in which the position of the medical device is indicated. However in this configuration position determination unit PDU is configured to compute the position of medical device MD based on ultrasound signals generated by the at least three ultrasound emitters attached to ultrasound imaging probe UIP, which are subsequently detected by polarized transducers PT1, PT2; i.e. transmitted between ultrasound imaging probe UIP and medical device MD. In this configuration position determination unit PDU determines the distance between each emitter and the mean position of polarized transducers PT1, PT2 based on the time of flight of ultrasound signals emitted by each emitter. The mean position of polarized transducers PT1, PT2 is subsequently determined using triangulation. This provides the mean position of polarized transducers PT1, PT2 in three dimensions respective ultrasound imaging probe UIP, and thus respective its ultrasound field UF since the at least three emitters are attached to the ultrasound imaging probe UIP.

Thus, position determination unit PDU in FIG. 4 may be used in either of the above configurations to compute a mean position of polarized transducers PT1, PT2 respective ultrasound field UF based on ultrasound signals transmitted between ultrasound imaging probe UIP and medical device MD. The mean position corresponds to the center-of-sensitivity of polarized transducers PT1, PT2.

In both these configurations, icon providing unit IPU in FIG. 4 is configured to provide, in the reconstructed image RUI, an icon IK indicating the position of medical device MD respective the ultrasound field UF. The icon may be for example a circle, a cross, a pointer and so forth and may for example be provided in the reconstructed image RUI using image fusion, an overlay, or by changing the contrast or color of the reconstructed ultrasound image RUI at the desired position of the icon or by using similar image fusion techniques.

Icon providing unit IPU may for example be implemented by means of a processor. Moreover, the function of any of the icon providing unit IPU, position determination unit PDU, or the image reconstruction unit IRU may be provided by one or more processors. These processors may include instructions configured to perform their respective functions outlined above. Such instructions may be included on a data carrier. Moreover, one or more of these units may be provided by imaging system processor ISP of ultrasound imaging system UIS.

Polarized transducers PT1, PT2 may in general be provided by discrete electronic components. These may then be attached to a medical device as described in relation to FIG. 3 and FIG. 4. In another configuration polarized transducers PT1, PT2 may be formed from a foil such as PVDF. Such a foil offers flexibility and is thus well suited to being attached to non-flat objects such as the shaft of a medical needle. Alternatively a PVDF polarized transducer may be formed using a dip coating process such as that disclosed in patent application WO2015155645. In another configuration described below, a transducer laminate may be provided that includes polarized transducers PT1, PT2.

FIG. 5 illustrates a transducer laminate TL that may be attached to a shaft of a medical device. FIG. 5A illustrates transducer laminate TL in plan view. FIG. 5B and FIG. 5C illustrate a section view along X-X′ in an assembled view and in an exploded view respectively. FIG. 5D and FIG. 5E illustrate a section view along Y-Y′ in an assembled view and in an exploded view respectively. FIG. 5F and FIG. 5G illustrate a section view along Z-Z′ in an assembled view and in an exploded view respectively. Transducer laminate TL in FIG. 5 includes first elongate foil F1, second elongate foil F2, first electrical conductor EC1, second electrical conductor EC2, first polarized transducer PT1 for detecting ultrasound signals, and second polarized transducer PT2 for detecting ultrasound signals. The first elongate foil F1, the second elongate foil F2, the first electrical conductor EC1 and the second electrical conductor EC2 each extend along length axis LAX. Transducer laminate TL may optionally include electrical shield ES to further reduce EMI. Electrical shield ES may be arranged to cover at least a portion of first electrical conductor EC1 and second electrical conductor EC2, and optionally may also cover part or all of the outer surfaces of first and second polarized transducers PT1, PT2. Electrical shield ES may be formed from a range of conductive materials such as metals, e.g. gold, aluminium, chrome and the like, or from a conductive polymer.

Polarized transducers PT1, PT2 and electrical conductors EC1, EC2 illustrated in FIG. 5A are connected together in the form of electrical circuit CCT1 of FIG. 1A. The polarization of each of polarized transducers PT1, PT2 is indicated by the +and symbols. The interconnection between polarized transducers PT1 and PT2 is made by conductive track CTR. Clearly other electrical circuits such as CCT2 of FIG. 1B may be implemented in a similar manner.

As indicated in FIG. 5B, at position X-X′ along length axis LAX first electrical conductor EC1, second electrical conductor EC2, first polarized transducer PT1 and second polarized transducer PT2 are sandwiched between first elongate foil F1 and second elongate foil F2. Moreover, first polarized transducer PT1 and second polarized transducer PT2 are arranged adjacent to one another; i.e. in a side-by-side configuration, and such that their outer faces that face the first elongate foil F1 have opposite polarity. Whilst in FIG. 5, PT1 and PT2 are illustrated as extending along the length axis LAX, other shapes of transducers and other arrangements in which first polarized transducer PT1 and second polarized transducer PT2 are adjacent to one another are also possible. These include separating PT1 and PT2 along length axis LAX. For example PT1 and PT2 may be arranged diagonally or at approximately 90 degrees thereto. Corresponding changes to the routing of conductive track CTR and any necessary electrical isolation to achieve the desired electrical circuit may also be used. These may be used to provide a desired transducer arrangement when transducer laminate TL is attached to a device. For example if transducer laminate TL in FIG. 5 is folded around an axis of the device about length axis LAX, then transducers PT1, PT2 are separated around the circumference of the device axis. In another example if transducer laminate TL is wrapped around an axis of the device then the transducers may be arranged in the form of a spiral around the circumference of the device axis. By adjusting the orientation of PT1 and PT2 and selecting the most appropriate wrapping/folding form of attachment, a high degree of flexibility in transducer arrangement is provided. Returning to FIG. 5B, first polarized transducer PT1 and second polarized transducer PT2 are connected between first electrical conductor EC1 and second electrical conductor EC2 electrically in series and with the same polarity as in CCT1 in FIG. 1A. Alternatively first polarized transducer PT1 and second polarized transducer PT2 could be connected between first electrical conductor EC1 and second electrical conductor EC2 electrically in parallel and with the same polarity, as in CCT2 in FIG. 1B. FIG. 5B also indicates first adhesive layer AL1 and second adhesive layer AL2 that may optionally be used to bond foil F1 and foil F2 together. A single adhesive layer, or no adhesive layer at all may also be used, the latter using Van der Waals forces to attach the two foils together. Optionally transducer laminate may include one or both of adhesive layers AL1A, AL2A disposed on the outer surfaces of transducer laminate TL in order to bond transducer laminate TL to a surface. As indicated for PT1 in FIG. 5B, first polarized transducer PT1 and second polarized transducer PT2 may comprise a polarized material layer PI, together with electrodes ELA, ELB that provide electrical contact with polarized material layer PI. As indicated in FIG. 5C in exploded view, along section X-X′ transducer laminate TL may be assembled by sandwiching electrical conductors EC1, EC2 between first elongate foil F1 and second elongate foil F2. First polarized transducer PT1, second polarized transducer PT2 and conductive track CTR are shown as being pressed into first adhesive layer AL1. Such a construction holds conductive track CTR in electrical contact with first polarized transducer PT1 and second polarized transducer PT2.

As indicated in FIG. 5D and FIG. 5E, at position Y-Y′ along length axis LAX, first electrical conductor EC1, second electrical conductor EC2, first polarized transducer PT1 and second polarized transducer PT2 are sandwiched between first elongate foil F1 and second elongate foil F2. However, here there is no conductive track CTR in the sandwich. Thus, as indicated in FIG. 5D and FIG. 5E, conductive track CTR may cover only a portion of the surface area of first polarized transducer PT1 and second polarized transducer PT2.

As indicated in FIG. 5F, at position Z-Z′ along the length axis LAX first electrical conductor EC1 and second electrical conductor EC2 are sandwiched between the first elongate foil F1 and the second elongate foil F2 and neither the first polarized transducer PT1 nor the second polarized transducer PT2 are sandwiched between the first elongate foil F1 and the second elongate foil F2. Position Z-Z′ thus defines an electrical interconnect portion of transducer laminate TL.

First and second elongate foils F1, F2 in FIG. 5 may be formed from a range of polymer materials, for example Polyethylene terephthalate (PET), Polyimides (PI), or Polyamides (PA) may be used. Preferably the foils are formed from an electrically insulating material. Adhesive layers AL1, AL1A, AL2, AL2A may in principle be any adhesive layer, although a pressure sensitive adhesive, i.e. PSA, layer is preferred. Pressure sensitive adhesives are a class of materials that form an adhesive bond upon application of pressure. Advantageously, pressure sensitive adhesives provide a reliable bond and thereby a robust structure that is quick to assemble. Suitable pressure sensitive adhesives include product 2811CL made by the 3M corporation. These may be supplied as PSA-coated polymer sheets such as product 9019 supplied by the 3M corporation. PSA-coated polymer sheets are typically provided with a removable outer layer that is peeled away to reveal the adhesive layer and thereby protect the adhesive layer until its adhesive properties are required. Moreover the adhesive layers AL1, AL1A, AL2, AL2A are preferably formed from an electrically insulating material. Electrical conductors EC1, EC2 provide electrical contact with the polarized transducers PT1, PT2, or more specifically with their corresponding electrodes ELA, ELB. Suitable materials for the electrical conductors include metals, for example, gold, aluminium, copper, silver and chrome. Preferably the electrical conductors are in the form of a wire. A wire, which conventionally has a substantially circular cross section, provides a transducer laminate TL with high flexibility. Polarized transducers PT1, PT2 in FIG. 5 are configured to detect ultrasound signals. Preferably these are made from a piezoelectric material. Polyvinylidene fluoride, i.e. PVDF, or the related materials in the PVDF group including PVDF co-polymers such as polyvinylidene fluoride trifluoroethylene, and PVDF ter-polymers such as P(VDF-TrFE-CTFE) are preferred materials for polarized transducers PT1, PT2 in FIG. 5. These materials are available in the form of a flexible layer that is easily incorporated into transducer laminate TL.

Transducer laminate TL in FIG. 5 may be attached to the shaft of a medical device. The medical device may be used in a tracking system that such as the above-described position tracking system described with reference to FIG. 4. Transducer laminate may for example be wrapped around the shaft of the medical device; for example wrapped around the shaft of the medical needle illustrated in FIG. 3.

In summary, a medical device that is less susceptible to EMI has been described. The medical device includes a body, a first electrical conductor, a second electrical conductor, a first polarized transducer, and a second polarized transducer. The first electrical conductor and the second electrical conductor each extend along the body. The first polarized transducer and the second polarized transducer are attached to the body such that their outer faces have opposite polarity. Moreover, the first polarized transducer and the second polarized transducer are connected between the first electrical conductor and second electrical conductor either i) electrically in series and with the same polarity; or ii) electrically in parallel and with the same polarity. In the medical device, a common EMI signal on each of the first electrical conductor and the second electrical conductor can subsequently be cancelled by subtracting the electrical signals on each of these conductors. Whilst the inventive medical device has been illustrated and described in detail in the drawings and foregoing description in relation to a position tracking system, this application is to be considered illustrative or exemplary and not restrictive. Moreover, the invention is not limited to the disclosed embodiments and can be used in various medical sensing applications. Moreover it is to be understood that the various examples and embodiments illustrated herein may be combined in order to provide various systems, devices and methods.

1. Transducer laminate for attachment to the shaft of a medical device;
the transducer laminate comprising:
a first elongate foil;
a second elongate foil;
a first electrical conductor;
a second electrical conductor;
a first polarized transducer for detecting ultrasound signals;
a second polarized transducer for detecting ultrasound signals;
wherein the first elongate foil, the second elongate foil, the first electrical conductor and the second electrical conductor each extend along a length axis;
wherein at a first position along the length axis the first electrical conductor, the second electrical conductor, the first polarized transducer and the second polarized transducer are sandwiched between the first elongate foil and the second elongate foil, and wherein the first polarized transducer and the second polarized transducer are arranged adjacent to one another and such that their outer faces that face the first elongate foil have opposite polarity, and wherein the first polarized transducer and the second polarized transducer are connected between the first electrical conductor and second electrical conductor either i) electrically in series and with the same polarity; or ii) electrically in parallel and with the same polarity; and
wherein at a second position along the length axis the first electrical conductor and the second electrical conductor are sandwiched between the first elongate foil and the second elongate foil and neither the first polarized transducer nor the second polarized transducer are sandwiched between the first elongate foil and the second elongate foil.

2. The transducer laminate according to claim 1 further comprising a differential amplifier circuit; wherein the differential amplifier circuit is electrically connected to the first electrical conductor and the second electrical conductor and is configured to generate an amplified difference electrical signal corresponding to an amplified difference between an electrical signal carried by the first electrical conductor and an electrical signal carried by the second electrical conductor.

3. Medical device comprising the transducer laminate according to claim 1.

4. Medical device according to claim 3; wherein the medical device includes a shaft, and wherein the transducer laminate is wrapped around the shaft.

5. Software-implemented method of discriminating between ultrasound signals and electromagnetic interference, the method comprising the steps of:
causing amplification, with a differential amplifier circuit, of a difference between an electrical signal carried by the first electrical conductor and an electrical signal carried by the second electrical conductor of the medical device of claim 3, wherein the first polarized transducer and the second polarized transducer are configured to detect ultrasound signals, to provide an amplified difference electrical signal;
causing conversion, with an analogue to digital converter circuit, of the amplified difference electrical signal into a digital signal.

6. A position tracking system comprising:
an ultrasound imaging probe;
an image reconstruction unit;
a position determination unit;
a medical device comprising:
a body;
a first electrical conductor;
a second electrical conductor;
a first polarized transducer configured to detect ultrasound signals;
a second polarized transducer configured to detect ultrasound signals;
wherein the first electrical conductor and the second electrical conductor each extend along the body;
wherein the first polarized transducer and the second polarized transducer are attached to the body such that their outer faces have opposite polarity;
wherein the first polarized transducer and the second polarized transducer are connected between the first electrical conductor and the second electrical conductor either i) electrically in series and with the same polarity; or ii) electrically in parallel and with the same polarity.
a differential amplifier circuit;
an icon providing unit;
wherein the ultrasound imaging probe is configured to generate and to detect ultrasound signals within an ultrasound field;
wherein the image reconstruction unit is configured to provide, based on the ultrasound signals generated by and detected by the ultrasound imaging probe a reconstructed ultrasound image corresponding to the ultrasound field;
wherein the differential amplifier circuit is electrically connected to the first electrical conductor and to the second electrical conductor of the medical device and is configured provide, in response to the detection of ultrasound signals transmitted between the ultrasound imaging probe and the medical device, an amplified difference electrical signal corresponding to an amplified difference between an electrical signal carried by the first electrical conductor and an electrical signal carried by the second electrical conductor,
wherein the position determination unit is configured to receive the amplified difference electrical signal, and to compute, based on the amplified difference electrical signal and based on the ultrasound signals transmitted between the ultrasound imaging probe and the medical device, a position of the medical device respective the ultrasound field; and
wherein the icon providing unit is configured to provide, in the reconstructed image, an icon indicating the position of the medical device respective the ultrasound field.

7. The position tracking system according to claim 6 wherein the medical device further comprises i) an electrical shield, wherein the electrical shield is configured to sandwich at least the first electrical conductor and the second electrical conductor between the electrical shield and the body; and/or ii) an insulator layer; wherein the insulator layer is disposed between the body and both the first polarized transducer and the second polarized transducer.

8. The position tracking system according to claim 6 wherein the body of the medical device has an elongate form.

9. The position tracking system according to claim 8 wherein the first electrical conductor and the second electrical conductor are each wrapped around the elongate body in the form of a spiral.

10. The position tracking system according to claim 9 wherein the first polarized transducer and the second polarized transducer are wrapped around the elongate body in the form of a ring.

11. The position tracking system according to claim 10 wherein the elongate body has an axis and wherein the first polarized transducer and the second polarized transducer are separated along the axis.

12. The position tracking system according to claim 6 wherein the body comprises a needle.

13. The position tracking system according to claim 6 wherein the first polarized transducer and the second polarized transducer are each formed from a piezoelectric material.

14. The position tracking system according to claim 6 wherein the first electrical conductor and the second electrical conductor are each formed from a wire.

15. The position tracking system according to claim 6 wherein the ultrasound signals transmitted between the ultrasound imaging probe and the medical device are either i) generated by the ultrasound imaging probe or ii) generated by at least three ultrasound emitters attached to the ultrasound imaging probe.
